(19) United States
(12) Patent Application Publication
(10) Pub. No.: US 2021/0161788 A1
(43) Pub. Date: Jun. 3, 2021

(54) Anti-Blue Light Dermal Topical Composition and Applications Thereof

(71) Applicant: Taiwan AC Scientific Inc., Taipei City (TW)

(72) Inventors: Chen-I Pang, Taipei City (TW); Wen-Hsin Pang, Taipei City (TW); Ya-Ching Tsui, Taipei City (TW); Shu-Fen Li, Taipei City (TW); Chi-Chun Pan, Taipei City (TW)

(21) Appl. No.: 16/821,233

(22) Filed: Mar. 17, 2020

(30) Foreign Application Priority Data
Nov. 29, 2019 (TW) 108143630

(51) Int. Cl. A61K 8/46; A61Q 17/04; A61K 8/60

(52) U.S. Cl. CPC A61K 8/466; A61K 8/602; A61Q 17/04

(57) ABSTRACT

The present disclosure provides an anti-blue light dermal topical composition which comprises terephthalylidene dicamphor sulfonic acid as an ingredient for effective anti-blue light and further rutoside for a better anti-blue light effect; the dermal topical composition contributes to skin health care and moderate skin damages attributed to blue light in environment; the present disclosure also provides an application of terephthalylidene dicamphor sulfonic acid to preparation of an anti-blue light medicament composition.

### BACKGROUND OF THE INVENTION

### 1. Field of the Invention

The present disclosure relates to skin health care, particularly a dermal topical composition and applications thereof to anti-blue light.

### 2. Description of the Prior Art

Electromagnetic waves known to the general public in daily lives are divided into ultraviolet (UV; wavelengths between 10 nm and 400 nm), visible light (VL; wavelengths between 400 nm and 700 nm) and infrared (IR; wavelengths between 700 nm and 1000 nm) according to wavelengths. As one electromagnetic wave, blue light (BL) with wavelengths between 400 nm and 500 nm is the visible light with strongest energy. People who soak up short-wavelength and high-energy blue light for a long period of time are likely to sustain cell injuries which cause dry skin, skin aging or melanin pigmentation. According to a research presented by University of Toledo, U.S.A., photoreceptor cells exposed to the environment full of blue light will be damaged by reactive oxygen from retinaldehyde.

Moreover, people operating more fancy and modern electronic products from day to day than before are chronically exposed to blue light emitted from electronic products and risk their health, for example, skin degradation. Anti-blue light products available in the market such as anti-blue light lens mostly do not work well as a protective apparatus for skin. Accordingly, how to find an ingredient with the anti-blue light effect and the potential of an application in a skin care product or cosmetic is the issue to be settled by the persons skilled in the art.

### SUMMARY OF THE INVENTION

In the present disclosure, terephthalylidene dicamphor sulfonic acid is chosen as an active candidate ingredient for anti-blue light and compared with rutin (also known as and hereinafter referred to as rutoside) which is a substance with the anti-blue light effect reportedly, for verifying the effect of terephthalylidene dicamphor sulfonic acid on anti-blue light. The structural formula of terephthalylidene dicamphor sulfonic acid is shown as follows:

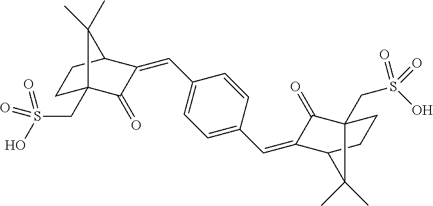 [SMILES: CC1(C)C2CCC1(CS(=O)(=O)O)C(=O)/C2=C\c1ccc(/C=C2\C(=O)C3(CS(=O)(=O)O)CCC2C3(C)C)cc1]

The present disclosure is to provide an anti-blue light dermal topical composition which comprises terephthalylidene dicamphor sulfonic acid with the anti-blue light effect.

To this end, the weight percentage of terephthalylidene dicamphor sulfonic acid in the dermal topical composition ranges from 0.1 to 25%.

To this end, the weight percentage of the terephthalylidene dicamphor sulfonic acid in the dermal topical composition can be 0.1%, 0.5%, 1%, 2%, 3%, 5%, 10%, 15%, 20%, 25%, 30%, 35%, 40%, 45%, 50%, 55%, 60%, 65%, 70%, 75% or more than 75%.

To this end, the dermal topical composition further comprises an adjuvant acceptable in skin care or cosmetology which can be a preservative, a humectant, a chelating agent or a neutralizer.

To this end, the dermal topical composition further comprises a preservative with a concentration between 0.1 and 3%, a humectant with a concentration between 1 and 10%, a chelating agent with a concentration between 0.001 and 1% and a neutralizer with a concentration between 0.05 and 6%.

To this end, the dermal topical composition further comprises rutoside which is paired with terephthalylidene dicamphor sulfonic acid for the synergistic effect of anti-blue light.

To this end, the ratio of the rutoside to the terephthalylidene dicamphor sulfonic acid in the dermal topical composition ranges from 1:0.5 to 1:2 or 1:1 preferably.

The present disclosure is to further provide an application of the dermal topical composition to skin health care wherein the skin health care involves moderation of skin damages attributed to blue light in environment.

To this end, the skin health care comprises moderated skin melanin pigmentations, fewer blotches on skin surfaces, skin whitening, prevention against dry skin, shrunk pores on skin surfaces, prevention against light aging, fine textured skin and reduced fine wrinkles.

The present disclosure is to further provide an application of terephthalylidene dicamphor sulfonic acid to preparation of an anti-blue light medicament composition wherein the medicament composition comprises terephthalylidene dicamphor sulfonic acid which is an active ingredient for anti-blue light.

To this end, the medicament composition is a topical liniment or topical medication and further comprises an adjuvant acceptable in pharmacology.

To this end, the medicament composition comprises rutoside which is taken as an active ingredient for anti-blue light and paired with terephthalylidene dicamphor sulfonic acid for the synergistic effect of anti-blue light.

In the present disclosure, a solution is offered for the issue of a dermal topical composition with the anti-blue light effect not available in the prior art in a long period. Specifically, the dermal topical composition in the present disclosure comprising an active ingredient of terephthalylidene dicamphor sulfonic acid is effective in blocking blue light, moderating skin damages induced by blue light and further inhibiting skin melanin pigmentations, blotches or fine wrinkles distributed on skin surfaces. The dermal topical composition in the present disclosure further comprises rutoside through which the anti-blue light effect is promoted.

These features and advantages of the present invention will be fully understood and appreciated from the following detailed description of the accompanying Drawings.

### BRIEF DESCRIPTION OF THE DRAWINGS

FIG. 1 illustrates results for the anti-blue light effect of terephthalylidene dicamphor sulfonic acid tested in a nail dryer.

FIG. 2 illustrates results for the anti-blue light effect of terephthalylidene dicamphor sulfonic acid tested with a blue-light laser pointer.

FIG. 3 illustrates curves for penetration rates of UV, blue light and visual light as well as the shading rate of blue light in terephthalylidene dicamphor sulfonic acid solutions with different concentrations.

FIG. 4 illustrates results for anti-blue light effects of rutoside and terephthalylidene dicamphor sulfonic acid tested in a nail dryer.

FIG. 5 illustrates shading rates of blue light compared between rutoside and terephthalylidene dicamphor sulfonic acid

FIG. 6 illustrates evaluations to the effect of terephthalylidene dicamphor sulfonic acid on skin melanin pigmentations at cheeks and the forehead.

FIG. 7 illustrates evaluations to the effect of terephthalylidene dicamphor sulfonic acid on skin melanin pigmentations at one side of a face.

FIG. 8 illustrates evaluations to the effect of terephthalylidene dicamphor sulfonic acid on fine wrinkles on skin surfaces.

### DETAILED DESCRIPTION OF THE PREFERRED EMBODIMENT

All technical and scientific terms in the patent specification have the same meanings as commonly understood by a person with ordinary skills in the art, unless otherwise specified.

The vocabularies of “a”, “one” and “the” for the singular form used in the patent specification are intended to include the plural forms as well, unless otherwise specified.

The vocabularies of “or”, “and” and “as well as” in the patent specification are intended to include the meaning of “and/or”, unless otherwise specified. Furthermore, the vocabularies of “comprise(s)/comprising” and “include(s)/including” should be considered as open transitional phrases not used to restrict the present invention. The descriptions in a previous paragraph are taken as the systematic references but not explained as restricting the present invention.

The term of “an adjuvant acceptable in pharmacology, skin care or cosmetology” means a substance or a composition compatible with other ingredients in a concoction and harmless to patients.

The adjuvant acceptable in pharmacology, skin care or cosmetology comprise one or several reagents selected from solvent, emulsifier, suspending agent, decomposer, binding agent, excipient, stabilizing agent, chelating agent, diluent, gelling agent, preservative, lubricant, surfactant, humectant, neutralizer or another similar adjuvant applicable to a dermal topical composition in the present disclosure.

The dosage form for an anti-blue light dermal topical composition and applications thereof to skin health care means, without limitation, an adjustable agent as required and a dermal topical agent preferably.

The dermal topical composition in the present disclosure means dermal topical agents such as ointments, lotions, emulsions, water aqua, packs, bath agents or creams and functions as a traditional dermal topical composition without limitation to a specific dosage form.

Moreover, in addition to active ingredients in the dermal topical composition, other ingredients in the form of dermal topical agents common in general cosmetics or medications can be properly added into bases such as humectant, antioxidant, oily constituent, UV absorber, surfactant, tackifier, alcohol, powder, toner, water-based constituent, water and skin nutrient. On the other hand, other substances to be added properly are shown as follows: metal blockers such as edetate disodium, edetate trisodium, sodium citrate, sodium polyphosphate, sodium metaphosphate and gluconic acid; medicaments such as caffeine, tannin, verapamil, tranexamic acid and derivatives thereof, crude drugs, tocopheryl acetate, glycyrrhizic acid and derivatives thereof or glycyrrhetate; other skin lighteners such as vitamin C, ascorbic phosphatase, ascorbyl glucoside, arbutin and kojic acid.

In the dermal topical composition mentioned above, one or multiple co-solvents, buffer agents, preservatives, colorants, spices or flavor enhancers can be properly added as required.

In the present disclosure, terephthalylidene dicamphor sulfonic acid, rutoside, medicaments, biological substances are available in the market.

The novel technical characteristics including specific characteristics in the present disclosure are disclosed in appended claims. The details of technical characteristics in the patent specification are explained in preferred embodiments based on theory in the present disclosure and drawings.

An anti-blue light dermal topical composition and applications thereof are elucidated in but not limited to the following embodiments.

### Embodiment 1, Test for the Anti-Blue Light Effect of Terephthalylidene Dicamphor Sulfonic Acid (1)

In this embodiment, the anti-blue light effect of terephthalylidene dicamphor sulfonic acid is tested in a nail dryer. Specifically, test panels on which the 1% terephthalylidene dicamphor sulfonic acid solution (experimental group) and the blank solution without terephthalylidene dicamphor sulfonic acid (control group) are instilled separately are irradiated in a nail dryer and checked for any color change after 20 seconds. The color on a test panel irradiated by a nail dryer from which UV light or blue light is launched may change to dark purple; the color on a test panel on which a solution with protective anti-UV or anti-blue light substances is covered will not change.

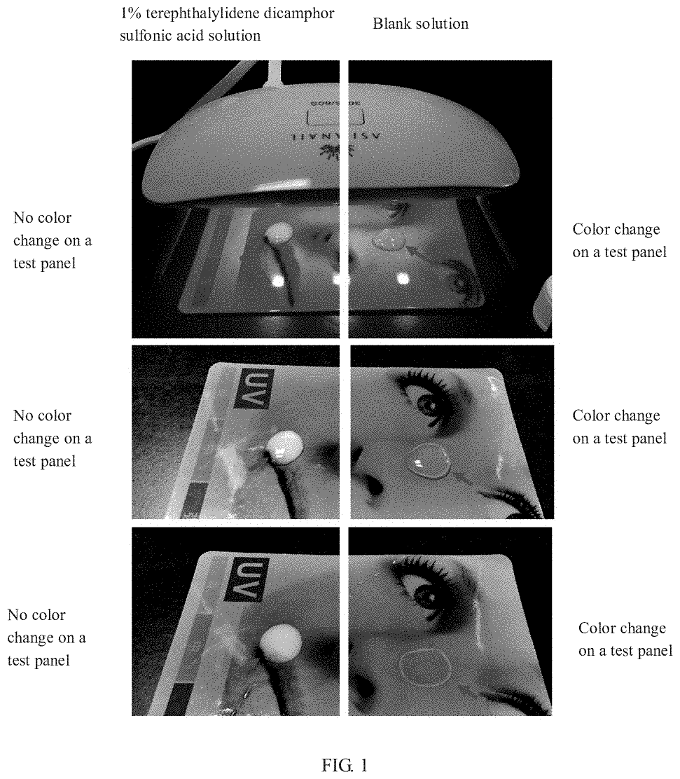

As shown in FIG. 1 and Table 1, the color on a test panel on which the 1% terephthalylidene dicamphor sulfonic acid solution is instilled locally does not change to dark purple during or after irradiation of a nail dryer. Thus, the anti-UV or anti-blue light effect of terephthalylidene dicamphor sulfonic acid is verified.

TABLE 1

| | Color change on a test panel | |
|---|---|---|
| | 1% terephthalylidene dicamphor | Blank |
| | sulfonic acid solution | solution |
| During irradiation | No color change | Color change |
| of a nail dryer | | |
| After irradiation | No color change | Color change |
| of a nail dryer | | |
| Nail dryer irradiating | No color change | Color change |
| & solution wiped | | |

### Embodiment 2, Test for the Anti-Blue Light Effect of Terephthalylidene Dicamphor Sulfonic Acid (2)

In this embodiment, the anti-blue light effect of terephthalylidene dicamphor sulfonic acid is tested with a 100 mW blue-light laser pointer. Specifically, transparent glass cups in which the 3% terephthalylidene dicamphor sulfonic acid solution (experimental group) and the blank solution without terephthalylidene dicamphor sulfonic acid (control group) are filled separately are irradiated with a 100 mW blue-light laser pointer. The status of a solution penetrated by blue light is checked with a piece of white paper on which any residual light is casted: a solution proves effective in blocking blue light if white light rather than blue light is casted on the piece of white paper.

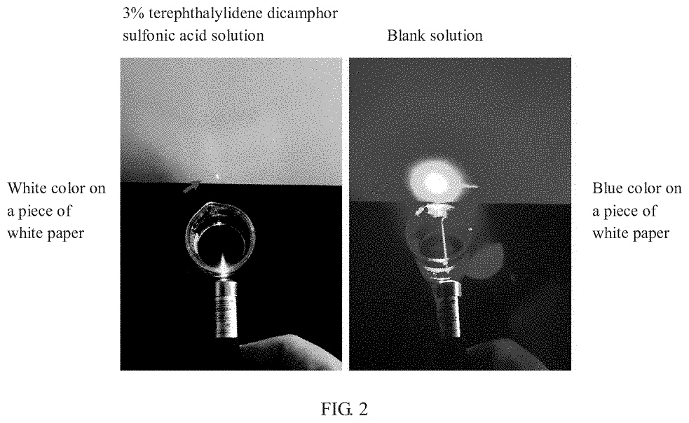

As shown in FIG. 2 and Table 2, the blank solution rather than the 3% terephthalylidene dicamphor sulfonic acid solution is easily penetrated by blue light. Thus, the anti-blue light effect of terephthalylidene dicamphor sulfonic acid is verified.

TABLE 2

| | 3% terephthalylidene dicamphor | Blank |
|---|---|---|
| | sulfonic acid solution | solution |
| Color locally shown on a | White | Blue |
| piece of white paper | | |

### Embodiment 3, Test for the Shading Rate of Blue Light by Terephthalylidene Dicamphor Sulfonic Acid Solutions with Different Concentrations

Solutions with different concentrations prepared by terephthalylidene dicamphor sulfonic acid are tested with a UV/blue-light detector (Actor-Mater Co., Ltd.) for penetration rates of ultraviolet (UV), blue light (BL) and visual light (VL). The shading rate of blue light is calculated by the formula as follows:

Shading rate of blue light (%)=penetration rate of visual light−penetration rate of blue light

where wavelength ranges of UV, blue light and visual light tested with a UV/blue-light detector (Actor-Mater Co., Ltd.) are 380-400 nm, 405-425 nm and 520-540 nm, respectively.

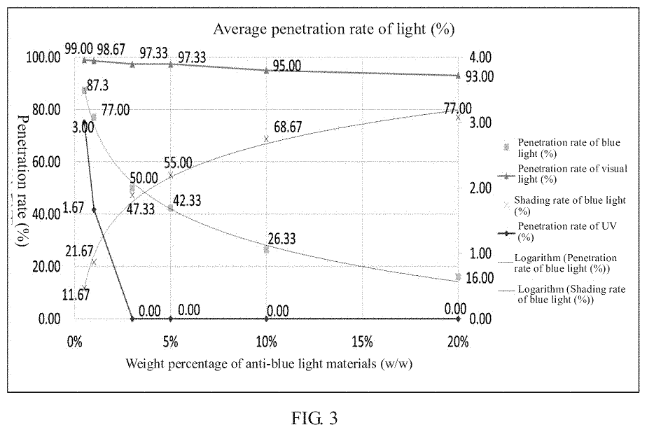

As shown in Table 3 and FIG. 3, low-concentration terephthalylidene dicamphor sulfonic acid proves effective in blocking blue light; the effect to block blue light is promoted with the concentration of a terephthalylidene dicamphor sulfonic acid solution increasing.

TABLE 3

| | | Concentrations of terephthalylidene | | | | | |
|---|---|---|---|---|---|---|---|
| | | dicamphor sulfonic acid solutions (W/W) | | | | | |
| | | 0.5% | 1% | 3% | 5% | 10% | 20% |
| Penetration | UV | 3.00 ± 0.00 | 1.67 ± 0.58 | 0.00 ± 0.00 | 0.00 ± 0.00 | 0.00 ± 0.00 | 0.00 ± 0.00 |
| rate (%) | BL | 87.33 ± 0.58 | 77.00 ± 1.00 | 50.00 ± 1.00 | 42.33 ± 0.58 | 26.33 ± 1.15 | 16.00 ± 1.00 |
| | VL | 99.00 ± 0.00 | 98.67 ± 0.58 | 97.33 ± 0.58 | 97.33 ± 0.58 | 95.00 ± 1.00 | 93.00 ± 1.00 |

| | | | | | | |
|---|---|---|---|---|---|---|
| Shading rate of | 11.67 ± 0.58 | 21.67 ± 0.58 | 47.33 ± 0.58 | 55.00 ± 1.00 | 68.67 ± 0.58 | 77.00 ± 0.00 |
| blue light (%) | | | | | | |

### Embodiment 4, Comparisons for Anti-Blue Light Effects Between Rutoside and Terephthalylidene Dicamphor Sulfonic Acid

The 3% terephthalylidene dicamphor sulfonic acid solution (B) and the 3% rutoside solution (C) are prepared by ingredients as shown in formulas in Table 4. Referring to test modes in Embodiment 1 to Embodiment 3, the anti-blue light effects and the shading rates of blue light for solutions (B) and (C) are tested, respectively.

TABLE 4

| | | 3% terephthalylidene | |
|---|---|---|---|
| | | dicamphor | |
| | Blank | sulfonic acid | 3% rutoside |
| Name | solution (A) | solution (B) | solution (C) |
| SymSave H | 0.50 | 0.50 | 0.50 |
| Hydrolite-5 | 2.00 | 2.00 | 2.00 |
| Elfamoist AC | 1.00 | 1.00 | 1.00 |
| Urea-Hydrovance | 1.00 | 1.00 | 1.00 |
| Zemea Propanediol | 1.00 | 1.00 | 1.00 |
| Water | 94.45 | 83.42 | 91.45 |
| Terephthalylidene | — | 9.38 | — |
| dicamphor | | | |
| sulfonic | | | |
| acid (32%) | | | |
| TEA- | — | 1.65 | — |
| Triethanolamine | | | |
| (99%) | | | |
| Rutoside | — | — | 3.00 |
| EDTA-2Na | 0.05 | 0.05 | 0.05 |

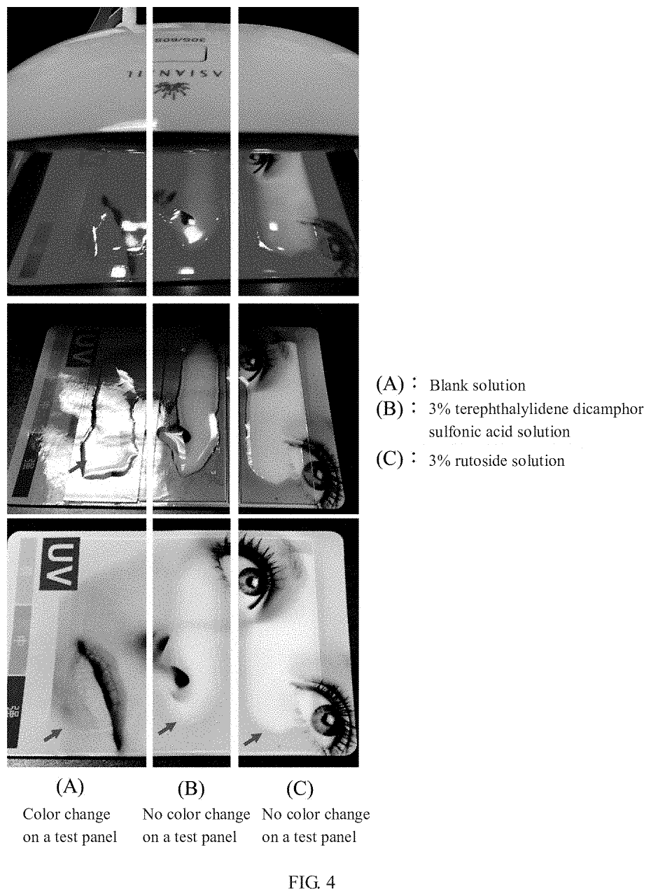

As shown in FIG. 4 and Table 5 for tests conducted in a nail dryer, both colors of test panels on which the 3% rutoside solution (C) and the 3% terephthalylidene dicamphor sulfonic acid solution (B) are instilled locally do not change to dark purple during or after irradiation of a nail dryer. Thus, the anti-UV or anti-blue light effect of the 3% terephthalylidene dicamphor sulfonic acid solution (B) or the 3% rutoside solution (C) is verified.

TABLE 5

| | Color change on a test panel | | |
|---|---|---|---|
| | | 3% terephthalylidene | |
| | | dicamphor | |
| | Blank | sulfonic acid | 3% rutoside |
| | solution (A) | solution (B) | solution (C) |
| During irradiation | Color | No color | No color |
| of a nail dryer | change | change | change |
| After irradiation | Color | No color | No color |
| of a nail dryer | change | change | change |
| Nail dryer | Color | No color | No color |
| irradiating | change | change | change |
| & solution wiped | | | |

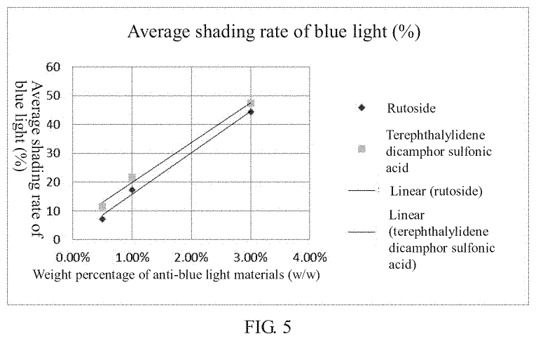

As shown in Table 6 and FIG. 5 for test results, the shading rate of blue light for terephthalylidene dicamphor sulfonic acid is higher than the shading rate of blue light for rutoside. Thus, the anti-blue light effect of terephthalylidene dicamphor sulfonic acid is better than that of rutoside.

TABLE 6

| | 0.5% | 1% | 3% |
|---|---|---|---|
| | Concentrations of rutoside solutions (W/W) | | |
| Shading rate of | 7.33 ± 0.58 | 17.33 ± 0.58 | 44.33 ± 0.58 |
| blue light (%) | | | |
| | Concentrations of terephthalylidene | | |
| | dicamphor sulfonic acid solutions (W/W) | | |
| Shading rate of | 11.67 ± 0.58 | 21.67 ± 0.58 | 47.33 ± 0.58 |
| blue light (%) | | | |

### Embodiment 5, Tests for the Synergistic Effect of Rutoside and Terephthalylidene Dicamphor Sulfonic Acid

The synergistic effect of rutoside and terephthalylidene dicamphor sulfonic acid on anti-blue light is explored in this embodiment. Prepared by rutoside and terephthalylidene dicamphor sulfonic acid, the mixed solutions with different concentrations are tested for shading rates of blue light according to the test mode as shown in Embodiment 3.

In a mixed solution, the ratio of rutoside to terephthalylidene dicamphor sulfonic acid ranges from 1:0.5 to 1:2 or is fixed to 1:1 preferably.

As shown in Table 7, the shading rates of blue light in mixed solutions prepared by rutoside and terephthalylidene dicamphor sulfonic acid are better that those of rutoside solutions or terephthalylidene dicamphor sulfonic acid solutions. Thus, the synergistic effect of rutoside and terephthalylidene dicamphor sulfonic acid on anti-blue light is verified.

TABLE 7

| | Average shading |
|---|---|
| | rate of blue light (%) |
| Content of anti-blue light substances | |
| in a solution (1%) | |
| 1% rutoside | 17 |
| 1% terephthalylidene dicamphor | 22 |
| sulfonic acid | |
| 0.5% rutoside & 0.5% terephthalylidene | 25 |
| dicamphor sulfonic acid | |
| Content of anti-blue light substances | |
| in a solution (3%) | |
| 3% rutoside | 44 |
| 3% terephthalylidene dicamphor | 47 |
| sulfonic acid | |
| 1.5% rutoside & 1.5% terephthalylidene | 52 |
| dicamphor sulfonic acid | |

### Embodiment 6, Evaluation to the Effect of Terephthalylidene Dicamphor Sulfonic Acid on Human Skin

Evaluation method: A subject who has applied the 3% terephthalylidene dicamphor sulfonic acid solution on skin surfaces for seven straight days is tested by the Janus facial analysis system for status change of his skin.

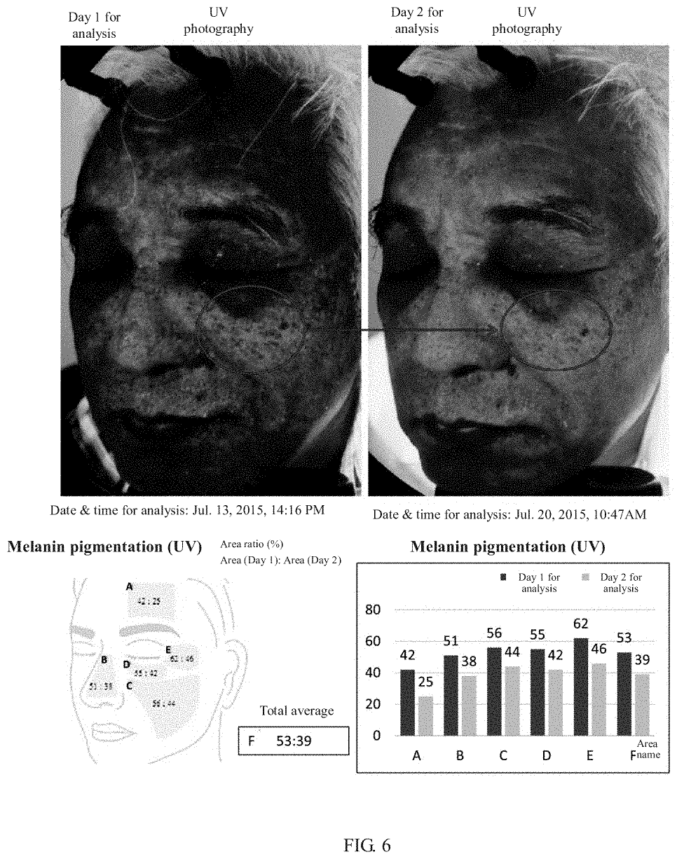

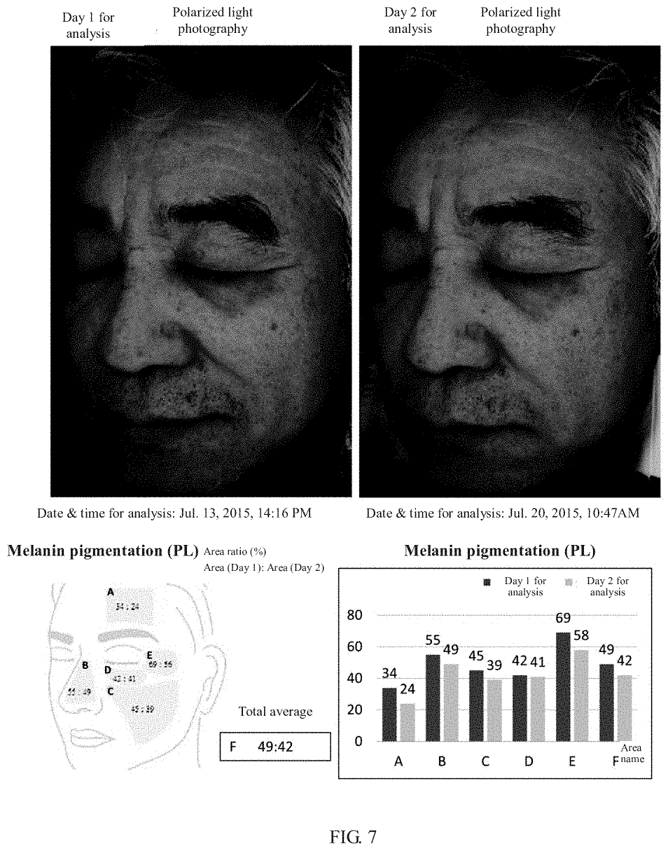

As shown in FIG. 6 and Table 8, the counts of melanin pigmentations at cheeks and the forehead of a subject who has applied the 3% terephthalylidene dicamphor sulfonic acid solution for seven straight days are reduced to 44 from 56 and 25 from 42, respectively. Furthermore, as shown in FIG. 7, the count of melanin pigmentations at one side of the face of a subject who has applied the 3% terephthalylidene dicamphor sulfonic acid solution for seven straight days is reduced to 42 from 49. It can be seen from test results that terephthalylidene dicamphor sulfonic acid proves effective in moderating melanin pigmentations or blotches on skin surfaces.

TABLE 8

| | Before | After 7 |
|---|---|---|
| | use | straight days |
| Count of melanin pigmentations | 56 | 44 |
| at cheeks | | |
| Count of melanin pigmentations | 42 | 25 |
| at the forehead | | |
| Count of melanin pigmentations | 49 | 42 |
| at one side of the face | | |

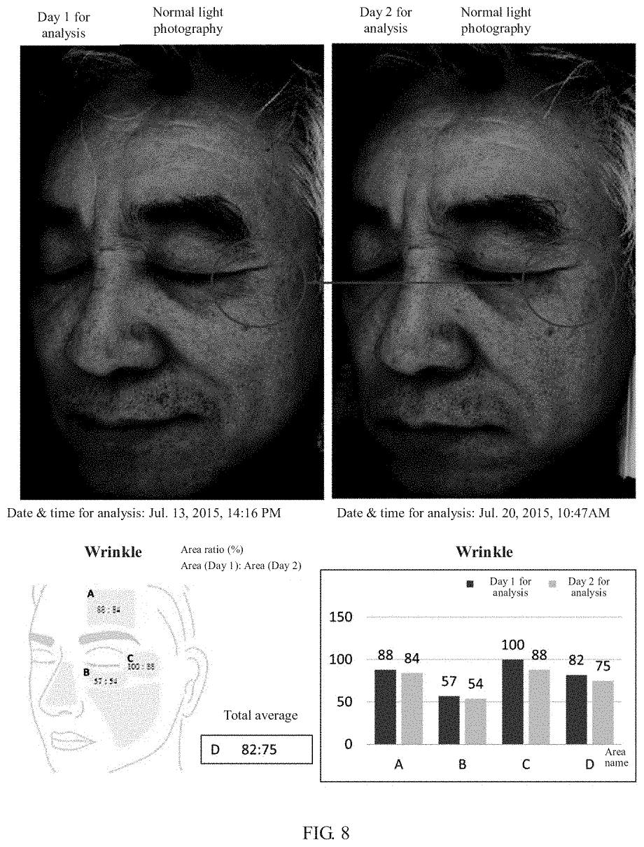

As shown in FIG. 8 and Table 9, the counts of fine wrinkles under eyelids and crow's feet of a subject who has applied the 3% terephthalylidene dicamphor sulfonic acid solution for seven straight days are reduced to 54 from 57 and 88 from 100, respectively; the total average of fine wrinkles is reduced by 7% (from 82 to 75). It can be seen from test results that terephthalylidene dicamphor sulfonic acid proves effective in moderating fine wrinkles on skin surfaces.

TABLE 9

| | Before | After 7 |
|---|---|---|
| | use | straight days |
| Fine wrinkles under eyelids | 57 | 54 |
| Crow's feet | 100 | 88 |
| Total average fine wrinkles | 82 | 75 |

The present disclosure provides innovative applications of terephthalylidene dicamphor sulfonic acid to the anti-blue light effect and moderation of skin damages attributed to blue light through a composition of terephthalylidene dicamphor sulfonic acid. Protecting skin from blue light, the composition spares skin from UV-induced injuries for skin whitening and reduced fine wrinkles. In the present disclosure, a further method to promote the anti-blue light effect of the composition is to add rutoside into the composition for the synergistic effect of rutoside and terephthalylidene dicamphor sulfonic acid on anti-blue light.

The descriptions presented in preferred embodiments of the patent specification are only examples clearly understood by a person with ordinary knowledge in the art; the embodiments can be implemented by the person with ordinary knowledge in the art through many modifications or changes of the embodiments without difference from technical characteristics of the present disclosure. Despite many modifications or changes based on embodiments of the patent specification, the present invention is still embodied. The scope of the present invention is defined as disclosed in appended claims and incorporates the above methods, architecture and other equivalent inventions.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. An anti-blue light dermal topical composition, comprising terephthalylidene dicamphor sulfonic acid with the anti-blue light effect.

2. The dermal topical composition as claimed in claim 1 wherein the terephthalylidene dicamphor sulfonic acid features a weight percentage equal to 0.1-25% of the dermal topical composition.

3. The dermal topical composition as claimed in claim 1 wherein the dermal topical composition further comprises an adjuvant acceptable in skin care or cosmetology which can be a preservative, a humectant, a chelating agent or a neutralizer.

4. The dermal topical composition as claimed in claim 1 wherein the dermal topical composition can be toning lotions, emulsions, skin creams, moisturizers, lip balms, essences, packs or facial cleansers.

5. The dermal topical composition as claimed in claim 1 wherein the dermal topical composition further comprises rutoside which is paired with terephthalylidene dicamphor sulfonic acid for the synergistic effect on anti-blue light.

6. The dermal topical composition as claimed in claim 2 wherein the dermal topical composition further comprises rutoside which is paired with terephthalylidene dicamphor sulfonic acid for the synergistic effect on anti-blue light.

7. The dermal topical composition as claimed in claim 3 wherein the dermal topical composition further comprises rutoside which is paired with terephthalylidene dicamphor sulfonic acid for the synergistic effect on anti-blue light.

8. The dermal topical composition as claimed in claim 4 wherein the dermal topical composition further comprises rutoside which is paired with terephthalylidene dicamphor sulfonic acid for the synergistic effect on anti-blue light.

9. The dermal topical composition as claimed in claim 5 wherein the ratio of the rutoside to the terephthalylidene dicamphor sulfonic acid in the dermal topical composition ranges from 1:0.5 to 1:2.

10. The dermal topical composition as claimed in claim 6 wherein the ratio of the rutoside to the terephthalylidene dicamphor sulfonic acid in the dermal topical composition ranges from 1:0.5 to 1:2.

11. The dermal topical composition as claimed in claim 7 wherein the ratio of the rutoside to the terephthalylidene dicamphor sulfonic acid in the dermal topical composition ranges from 1:0.5 to 1:2.

12. The dermal topical composition as claimed in claim 8 wherein the ratio of the rutoside to the terephthalylidene dicamphor sulfonic acid in the dermal topical composition ranges from 1:0.5 to 1:2.

13. A method of a skin care comprising:
administering an dermal topical composition to an individual in need thereof;
wherein the dermal topical composition comprising a terephthalylidene dicamphor sulfonic acid which has an anti-blue light effect; and
wherein the skin treatment involves moderation of skin damages attributed to blue light in environment.

14. The method as claimed in claim 13, wherein the skin care comprises moderating skin melanin pigmentations, lowering blotches on skin surfaces, skin whitening, prevention against dry skin, shrinking pores on skin surfaces, prevention against photoaging, improving textured skin or reducing fine wrinkles.

15. The method claimed in claim 13, wherein the dermal topical composition is a pharmaceutical composition.

16. The method as claimed in claim 15, wherein the pharmaceutical composition further comprises an adjuvant acceptable in pharmacology.

17. The method as claimed in claim 13, wherein the dermal topical composition further comprises a rutoside which is an active ingredient for anti-blue light and paired with terephthalylidene dicamphor sulfonic acid for the synergistic effect on anti-blue light.

18. The method as claimed in claim 14 wherein the dermal topical composition further comprises a rutoside which is an active ingredient for anti-blue light and paired with terephthalylidene dicamphor sulfonic acid for the synergistic effect on anti-blue light.
